(19) United States
(12) Patent Application Publication
(10) Pub. No.: US 2021/0008234 A1
(43) Pub. Date: Jan. 14, 2021

(54) ULTRAVIOLET STERILIZER

(71) Applicant: Enplas Corporation, Saitama (JP)

(72) Inventor: Masato NAKAMURA, Saitama (JP)

(21) Appl. No.: 16/969,948

(22) PCT Filed: Feb. 13, 2019
(86) PCT No.: PCT/JP2019/005151

§ 371 Date: Aug. 13, 2020

(30) Foreign Application Priority Data
Feb. 14, 2018 (JP) 2018-023989

(51) Int. Cl. A61L 2/10; A61L 2/26

(52) U.S. Cl. CPC A61L 2/10; A61L 2202/20; A61L 2202/11; A61L 2/26

(57) ABSTRACT

The ultraviolet sterilizer according to the present invention has: a flow channel pipe which has a treatment flow channel therein; a light source which emits ultraviolet rays; a condensing lens which condenses, toward the treatment flow channel, a portion of the ultraviolet rays emitted from the light source; and a reflector which reflects, toward the treatment flow channel, another portion of the ultraviolet rays emitted from the light source.

### TECHNICAL FIELD

The present invention relates to an ultraviolet sterilization apparatus.

### BACKGROUND ART

It is well known to sterilize fluids such as liquids and gasses with ultraviolet radiation. For example, Patent Literature (hereinafter, referred to as “PTL”) 1 describes a fluid sterilization apparatus irradiating a channel that extends in the axial direction of the apparatus with ultraviolet radiation in the axial direction to sterilize a liquid or a gas flowing through the channel.

Specifically, the fluid sterilization apparatus described in PTL 1 includes a channel pipe that demarcates a treatment channel extending in the axial direction, and a wide-orientation-angle light emitting element (LED light source) that is disposed in the vicinity of one end of the channel pipe and radiates ultraviolet radiation in the axial direction from the one end toward the treatment channel. The ultraviolet radiation radiated from the light emitting element sterilizes a fluid inside the treatment channel.

### CITATION LIST

### Patent Literature

PTL 1
Japanese Patent Application Laid-Open No. 2017-104230

### SUMMARY OF INVENTION

### Technical Problem

Liquids such as water typically absorb ultraviolet radiation. Ultraviolet radiation emitted from an ultraviolet radiation emitting element is generally divergent light, so that the illuminance of the ultraviolet radiation decreases in inverse proportion to the square of the distance from the light source. The ultraviolet radiation shows lowering of its illuminance as the distance from the light emitting element increases. When the flow rate of the liquid is constant, the liquid close to the light emitting element is easily sterilized compared with the liquid far from the light emitting element because the liquid close to the light emitting element is less affected by the absorption of ultraviolet radiation. Increasing a sterilizing effect requires enhancement of the illuminance of ultraviolet radiation in the vicinity of the light emitting element. The number of light emitting elements may be increased to enhance the illuminance of ultraviolet radiation in the vicinity of the light emitting element. However, increasing light emitting elements number may also increase the manufacturing cost and the size of the apparatus.

An object of the present invention is to provide an ultraviolet sterilization apparatus capable of increasing a sterilization effect without increasing the number of light sources.

### Solution to Problem

An ultraviolet sterilization apparatus of the present invention directed to achieve the above object is an ultraviolet sterilization apparatus for sterilizing a fluid flowing through a treatment channel by irradiating the fluid with ultraviolet radiation, the ultraviolet sterilization apparatus including: a channel pipe including therein the treatment channel; a light source configured to emit the ultraviolet radiation; a condenser lens configured to condense, toward the treatment channel, a part of the ultraviolet radiation emitted from the light source; and a reflector that reflects another part of the ultraviolet radiation, toward the treatment channel, emitted from the light source.

### Advantageous Effects of Invention

The present invention is capable of increasing the sterilization effect without increasing the number of light sources.

### BRIEF DESCRIPTION OF DRAWINGS

FIG. 1 is a cross-sectional view of an ultraviolet sterilization apparatus according to embodiment 1 of the present invention;

FIGS. 2A to 2D illustrate a configuration of a condenser lens according to embodiment 1;

FIGS. 3A to 3D illustrate a configuration of a reflector according to embodiment 1;

FIGS. 4A to 4C illustrate optical paths in the ultraviolet sterilization apparatus according to embodiment 1;

FIG. 5 is a cross-sectional view of an ultraviolet sterilization apparatus according to a modification;

FIGS. 6A to 6D illustrate a configuration of a reflector according to the modification;

FIGS. 7A to 7C illustrate optical paths in the ultraviolet sterilization apparatus according to the modification;

FIG. 8 is a cross-sectional view of an ultraviolet sterilization apparatus according to embodiment 2 of the present invention;

FIGS. 9A to 9D illustrate a configuration of a condenser lens according to embodiment 2;

FIGS. 10A to 10D illustrate a configuration of a reflector according to embodiment 2;

FIGS. 11A to 11C illustrate optical paths in the ultraviolet sterilization apparatus according to embodiment 2;

FIG. 12 is a cross-sectional view of an ultraviolet sterilization apparatus according to a modification;

FIGS. 13A to 13D illustrate a configuration of a reflector according to the modification; and

FIGS. 14A to 14C illustrate optical paths in the ultraviolet sterilization apparatus according to the modification.

### DESCRIPTION OF EMBODIMENT

Embodiments of the present invention will be described in detail below with reference to the accompanying drawings.

### Embodiment 1

(Configuration of Ultraviolet Sterilization Apparatus)

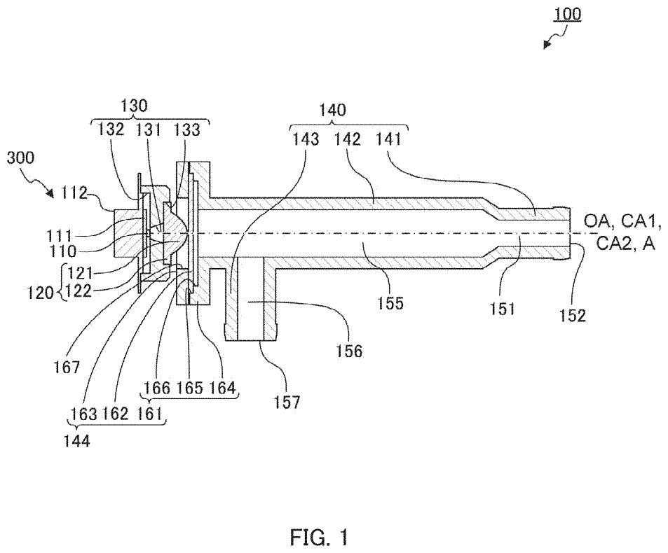

FIG. 1 is a cross-sectional view of ultraviolet sterilization apparatus 100 according to embodiment 1 of the present invention.

As illustrated in FIG. 1, ultraviolet sterilization apparatus 100 includes light source 110, condenser lens 120, reflector 130 and channel pipe 140 including treatment channel 155. Light source 110, condenser lens 120 and reflector 130 function as ultraviolet irradiation apparatus 300.

(Light Source)

Light source 110 emits ultraviolet radiation toward treatment channel 155. The type of light source 110 is not particularly limited as long as it can emit ultraviolet radiation. Examples of light source 110 include light emitting diodes (LEDs), mercury lamps, metal halide lamps, xenon lamps and laser diodes (LDs). The central wavelength or peak wavelength of ultraviolet radiation emitted from light source 110 is preferably 200 nm or more and 350 nm or less. The central wavelength or peak wavelength of the ultraviolet radiation emitted from light source 110 is more preferably 260 nm or more and 290 nm or less from the viewpoint of high sterilization efficiency. Light source 110 preferably has a wide orientation angle, and preferably is, for example, an LED having 60° or more of a directional half width, an angle between directions having the intensity of brightness that becomes 50% of the peak value.

Light source 110 is disposed on one surface of substrate 111. The other surface of substrate 111 is attached to the central portion of base 112. Reflector 130 is disposed around substrate 111 disposed on base 112.

(Configuration of Condenser Lens)

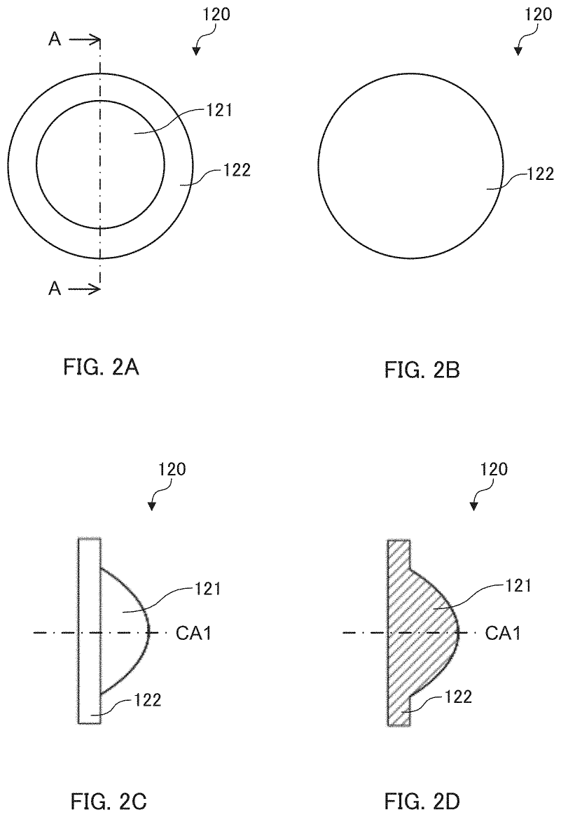

FIGS. 2A to 2D illustrate a configuration of condenser lens 120. FIG. 2A is a plan view of condenser lens 120, FIG. 2B is a bottom view thereof, FIG. 2C is a side view thereof and FIG. 2D is a cross-sectional view taken along line A-A shown in FIG. 2A.

Condenser lens 120 condenses, toward treatment channel 155, part of the ultraviolet radiation (ultraviolet radiation having a small emission angle) emitted from light source 110. In addition, condenser lens 120 condenses, toward treatment channel 155, part of the ultraviolet radiation (ultraviolet radiation having a large emission angle) emitted from light source 110 and reflected by reflection surface 131. As illustrated in FIG. 2A to 2D, condenser lens 120 includes convex lens surface 121 and flange 122.

Convex lens surface 121 is disposed on the channel pipe 140 side or on the light source 110 side. That is, convex lens surface 121 may be disposed on the channel pipe 140 side or on the light source 110 side, or may be disposed on both the channel pipe 140 side and the light source 110 side. In the present embodiment, convex lens surface 121 is disposed only on the channel pipe 140 side, and the light source 110 side is in a shape of a flat surface (plane surface). The flat surface of condenser lens 120 on the light source 110 side functions as an incidence surface for receiving the ultraviolet radiation emitted from light source 110, and convex lens surface 121 of condenser lens 120 on the channel pipe 140 side functions as an emission surface for emitting the ultraviolet radiation traveling inside condenser lens 120.

In the present embodiment, convex lens surface 121 is circularly symmetrical with first central axis CA1 as a rotation axis. In a cross section including first central axis CA1, convex lens surface 121 is formed so that the diameter thereof decreases in cross sections perpendicular to first central axis CA1 from the light source 110 side toward channel pipe 140 side. In addition, it is preferable that optical axis OA of light source 110 and first central axis CA1 of condenser lens 120 are coincident with each other.

Flange 122 is disposed around convex lens surface 121. Flange 122 not only simplifies the handling of condenser lens 120, but also functions as an installation portion to reflector 130.

In the present embodiment, part of the ultraviolet radiation emitted from light source 110 is refracted toward channel pipe 140 as the part is incident on the flat surface on the light source 110 side and emitted from convex lens surface 121.

(Configuration of Reflector)

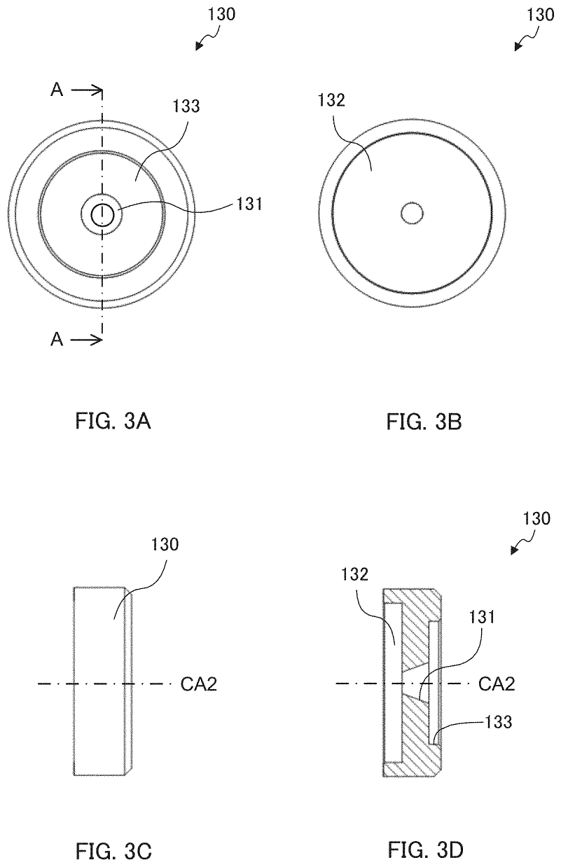

FIGS. 3A to 3D illustrate a configuration of reflector 130. FIG. 3A is a plan view of reflector 130, FIG. 3B is a bottom view thereof, FIG. 3C is a side view thereof and FIG. 3D is a cross-sectional view taken along line A-A shown in FIG. 3A.

Reflector 130 reflects part of the ultraviolet radiation emitted from light source 110, i.e., ultraviolet radiation having a large emission angle, toward treatment channel 155. As illustrated in FIGS. 3A to 3D, reflector 130 includes reflection surface 131, first recess 132 and second recess 133.

Reflection surface 131 reflects ultraviolet radiation, which is emitted from light source 110 and directly reaches reflection surface 131, toward treatment channel 155. Reflection surface 131 is circularly symmetrical with second central axis CA2 as a rotation axis. In addition, it is preferable that optical axis OA of light source 110, first central axis CA1 of condenser lens 120 and second central axis CA2 of reflector 130 are coincident with each other.

The shape of reflection surface 131 in a cross section including second central axis CA2 is not particularly limited. The shape of reflection surface 131 in the cross section including second central axis CA2 may be linear, or curved concave with respect to second central axis CA2. In the present embodiment, the shape of reflection surface 131 in the cross section including second central axis CA2 is linear.

First recess 132 is formed in the surface of reflector 130 on the light source 110 side. The central portion of the bottom of first recess 132 communicates with one end of reflection surface 131. In ultraviolet sterilization apparatus 100, first recess 132 houses light source 110 and substrate 111.

Second recess 133 is formed in the surface of reflector 130 on the channel pipe 140 side. The central portion of the bottom of second recess 133 communicates with one end of recess surrounded by reflection surface 131. In ultraviolet sterilization apparatus 100, condenser lens 120 is installed at second recess 133.

Disposing reflector 130 on base 112 allows first recess 132 to house light source 110 and substrate 111, and reflection surface 131 to surround light source 110.

(Configuration of Channel Pipe)

A fluid to be sterilized flows through channel pipe 140. Channel pipe 140 is formed of a material that is not easily deformed or broken by the pressure of a fluid flowing through treatment channel 155. Channel pipe 140 includes inlet pipe 141 having treatment channel 155 therein, inlet pipe body 142, outlet pipe 143 and incidence window 144.

Inlet pipe 141 is used to introduce a fluid, which is to be sterilized by irradiation with ultraviolet radiation, into treatment channel 155. Inlet pipe 141 includes therein inlet channel 151. The upstream end of the inlet pipe 141 is inlet port 152 for allowing a fluid to flow into inlet channel 151. The downstream end of inlet pipe 141 is open to the pipe wall at the upstream end of channel pipe body 142. Inlet pipe 141 is connected to an unillustrated fluid supply apparatus or the like via inlet port 152 to guide the fluid from the fluid supply apparatus to treatment channel 155. Inlet port 152 may have a shape such that a hose for guiding the fluid to inlet channel 151 can be fitted.

Channel pipe body 142 includes therein treatment channel 155 extending from one end side toward the other end side. The shape of the channel pipe body 142 is not particularly limited as long as fluid can flow. The shape of treatment channel 155 may be linear or curved. In the present embodiment, the shape of treatment channel 155 is linear. In addition, the cross-sectional shape of treatment channel 155 in a direction perpendicular to the fluid flowing direction is not particularly limited, either. The cross-sectional shape may be circular or polygonal. In the present embodiment, the cross-sectional shape of treatment channel 155 in the direction perpendicular to the fluid flowing direction is circular. In addition, it is preferable that optical axis OA of light source 110, first central axis CA1 of condenser lens 120, second central axis CA2 of reflector 130 and axis A of channel pipe body 142 (treatment channel 155) are coincident with each other.

Channel pipe body 142 is preferably formed of a material having high reflectance of ultraviolet radiation. Examples of materials for channel pipe body 142 include mirror polished aluminum (Al) and polytetrafluoroethylene (PTFE). The material of the channel pipe body 142 is preferably PTFE from the viewpoint of being chemically stable and also having a higher reflectance of ultraviolet radiation. Forming channel pipe body 142 from a material having a high reflectance of ultraviolet radiation enables increase of the utilization efficiency of ultraviolet radiation emitted from light source 110.

Channel pipe body 142 may be in any size as long as fluid can be sufficiently sterilized by irradiation with ultraviolet radiation. When, for example, only one light source 110 with a light output of 30 mW/lamp is used, channel pipe body 142 may have an inner diameter of 5 cm or less and a channel length of 2 cm or more and 30 cm or less. In the present embodiment, incidence window 144 is disposed at the downstream end surface.

Outlet pipe 143 is used to allow the sterilized fluid to flow out of treatment channel 155. Outlet pipe 143 includes therein outlet channel 156. The upstream end of outlet pipe 143 is open to the vicinity of the downstream end of channel pipe body 142. The downstream end of outlet pipe 143 is outlet port 157 for guiding fluid to an unillustrated liquid storage apparatus or the like. Outlet port 157 is connected to the liquid storage apparatus or the like and guides a fluid from treatment channel 155. Outlet port 157 may have a shape such that a hose for guiding the fluid to the liquid storage apparatus or the like can be fitted.

Incidence window 144 transmits ultraviolet radiation, which is emitted from light source 110 and reaches incidence window 144 via condenser lens 120, into channel pipe 140 (channel pipe body 142). The position where incidence window 144 is disposed is not particularly limited as long as the above described function is exhibited. In the present embodiment, incidence window 144 is disposed at the downstream end of channel pipe body 142. Incidence window 144 includes a transparent plate holding portion 161, a transparent plate 162, and fixing lid 163. Transparent plate holding portion 161 and fixing lid 163 may have a screw hole or the like for inserting a screw for joining them.

Transparent plate holding portion 161 includes outer wall portion 164, third recess 165, and fourth recess 166.

Outer wall portion 164 is formed integrally with channel pipe body 142. Outer wall portion 164 may have any size and strength as long as transparent plate 162 can be fixed.

Third recess 165 is a recess where transparent plate 162 is to be disposed. The shape of third recess 165 is not particularly limited as long as the shape is complementary to that of transparent plate 162. For example, transparent plate 162 in a rectangular plate shape makes third recess 165 into a recess in a rectangular shape in plan view. In addition, transparent plate 162 in a circular plate shape makes third recess 165 into a recess in a circular shape in plan view. Fourth recess 166 is formed in the central portion of the bottom of third recess 165.

Fourth recess 166 reduces the flow rate of fluid (to slow down) flowing through the treatment channel to mitigate the impact of the liquid on transparent plate 162. Fourth recess 166 may have any depth as long as fluid having flowed through treatment channel 155 can extend sufficiently in fourth recess 166.

Fixing lid 163 is a plate shaped member for fixing transparent plate 162 from the outside. Ultraviolet radiation transmitting hole 167 is formed in the central portion of fixing lid 163. Fixing of fixing lid 163 to outer wall portion 164 allows transparent plate 162 to be fixed between outer wall portion 164 and fixing lid 163.

Ultraviolet radiation transmitting hole 167 transmits ultraviolet radiation emitted from light source 110. The transmitted ultraviolet radiation reaches treatment channel 155 via transparent plate 162. Ultraviolet radiation transmitting hole 167 preferably has a shape capable of housing therein convex lens surface 121 of condenser lens 120 to reduce the distance between condenser lens 120 and transparent plate 162, thereby suppressing the loss of the amount of light while ultraviolet radiation emitted from condenser lens 120 propagates through the air.

Transparent plate 162 is formed of any material capable of transmitting ultraviolet radiation. The inner surface of transparent plate 162 functions as a part of outer periphery of the treatment channel 155 to prevent fluid from flowing out from one end of channel pipe body 142. Examples of materials for transparent plate 162 include materials with high transparency to ultraviolet radiation, such as quartz (SiO₂), sapphire (Al₂O₃), and amorphous fluorine resins.

A fluid introduced from inlet port 152 to treatment channel 155 via inlet channel 151 is sterilized by irradiation with ultraviolet radiation emitted from light source 110 while flowing through treatment channel 155. The fluid having sterilized is discharged from outlet port 157 via outlet channel 156.

The fluid may be any material that can flow through treatment channel 155 to be sterilized, and may be, for example, a liquid such as water. Examples of the fluid include tap water such as drinking water and agricultural water, and sewage such as drainage from factories.

The fluid may have any flow rate as long as the fluid is sufficiently sterilized by the irradiation with ultraviolet radiation while flowing through treatment channel 155. The flow rate of the fluid is, for example, 10 L/min or less when only one light source 110 with an output of 30 mW/lamp is used and the fluid is a liquid.

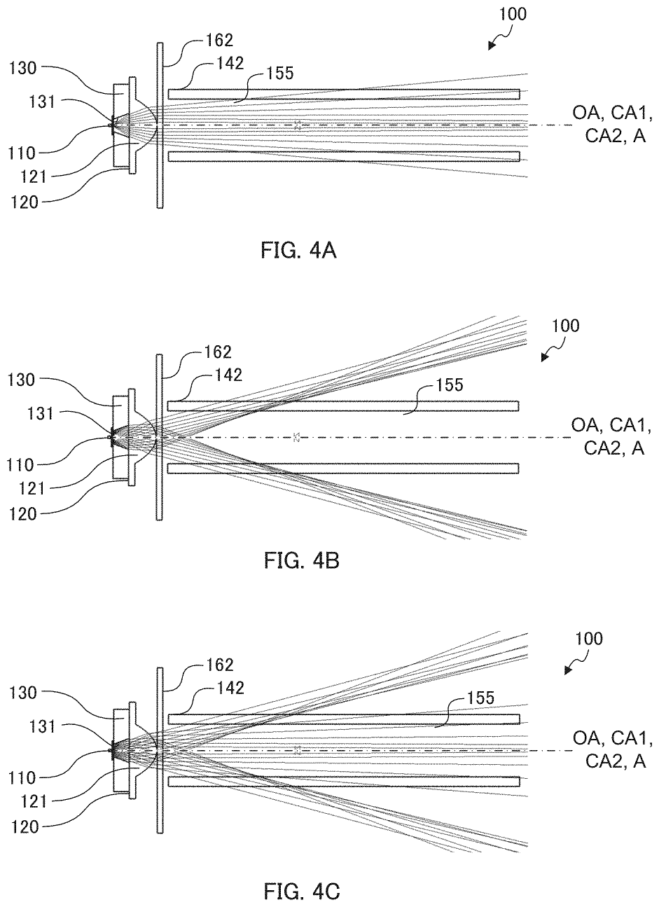

FIGS. 4A to 4C illustrate optical paths in ultraviolet sterilization apparatus 100. FIG. 4A illustrates optical paths of light emitted from light source 110 and controlled by condenser lens 120 without controlled by reflector 130, FIG. 4B illustrates optical paths of light emitted from light source 110 and controlled by reflector 130, and FIG. 4C illustrates optical paths combining the optical paths of FIGS. 4A and 4B. FIGS. 4A to 4C illustrate only light source 110, condenser lens 120, reflector 130 and treatment channel 155 to show the optical paths.

As illustrated in FIGS. 4A and 4C, part (also referred to as “first part”) of ultraviolet radiation emitted from light source 110, i.e., ultraviolet radiation having a small emission angle, directly reaches condenser lens 120 without controlled by reflector 130. The ultraviolet radiation reaching condenser lens 120 enters condenser lens 120 from the flat surface of condenser lens 120 on the light source 110 side. The ultraviolet radiation entering condenser lens 120 is emitted to the outside from convex lens surface 121 on the treatment channel 155 side. During the procedure, the ultraviolet radiation is refracted by convex lens surface 121 toward optical axis OA of light source 110. The ultraviolet radiation emitted from light source 110 at a small emission angle is refracted by condenser lens 120 so that the angle of the ultraviolet radiation with respect to optical axis OA becomes smaller. The ultraviolet radiation emitted from convex lens surface 121 radiates through transparent plate 162 into treatment channel 155. Since treatment channel 155 is irradiated with the ultraviolet radiation condensed by condenser lens 120, a certain level of high illuminance can be obtained not only in the vicinity of light source 110 but also in the region far from light source 110.

As illustrated in FIGS. 4B and 4C, another part (also referred to as “second part”) of ultraviolet radiation, i.e., ultraviolet radiation having a large emission angle, cannot reach condenser lens 120 unless the radiation is controlled. Accordingly, reflector 130 reflects the ultraviolet radiation emitted from light source 110 at a large emission angle toward condenser lens 120. As illustrated in FIGS. 4B and 4C, the second part of ultraviolet radiation emitted from light source 110, i.e., ultraviolet radiation having a large emission angle, reaches reflection surface 131 of reflector 130. The ultraviolet radiation reaching reflection surface 131 is reflected toward the incidence surface (flat surface) of condenser lens 120. The ultraviolet radiation reaching the incidence surface enters the condenser lens from the incidence surface and is emitted from convex lens surface 121. The ultraviolet radiation emitted from convex lens surface 121 radiates through transparent plate 162 into treatment channel 155. Additional irradiation of treatment channel 155 with the ultraviolet radiation having a large emission angle enhances the illuminance in treatment channel 155.

(Effects)

From the foregoing, ultraviolet sterilization apparatus 100 according to embodiment 1 includes condenser lens 120 and reflector 130 which condense ultraviolet radiation emitted from light source 110 and radiate the ultraviolet radiation toward the treatment channel. The illuminance in treatment channel 155 is thus enhanced.

[Modification]

Ultraviolet sterilization apparatus 100a of a modification of embodiment 1 will be described. Ultraviolet sterilization apparatus 100a of modification 1 differs from ultraviolet sterilization apparatus 100 according to embodiment 1 only in the configuration of reflector 130a. Therefore, the same components as those of ultraviolet sterilization apparatus 100 according to embodiment 1 are designated by the same reference numerals and the description thereof will be omitted.

(Configuration of Ultraviolet Sterilization Apparatus)

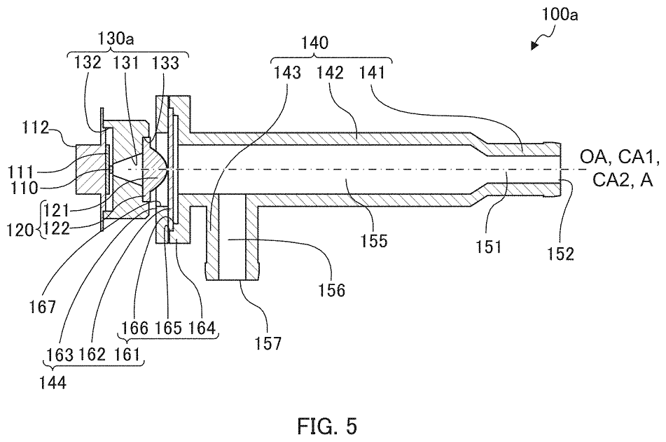

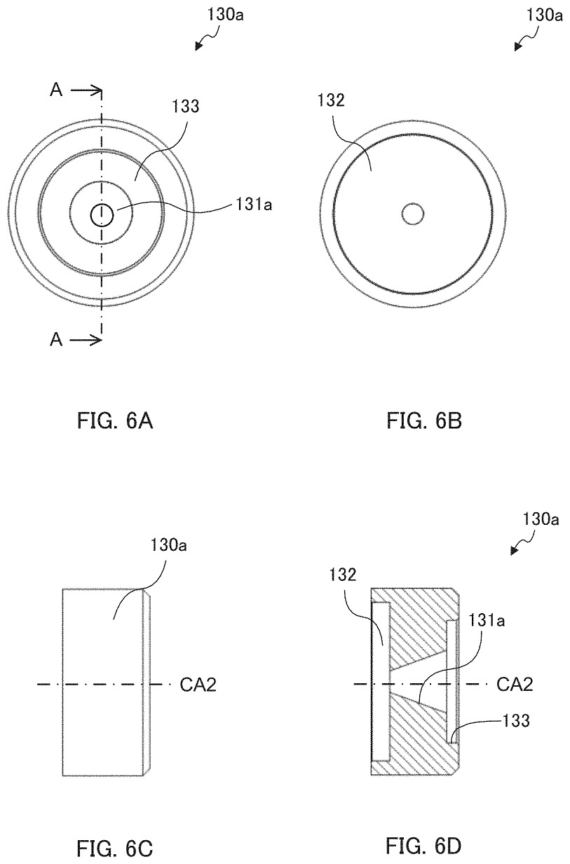

FIG. 5 is a cross-sectional view of ultraviolet sterilization apparatus 100a according to the modification. FIGS. 6A to 6D illustrate a configuration of reflector 130a. FIG. 6A is a plan view of reflector 130a, FIG. 6B is a bottom view thereof, FIG. 6C is a side view thereof and FIG. 6D is a cross-sectional view taken along line A-A shown in FIG. 6A.

As illustrated in FIG. 5, ultraviolet sterilization apparatus 100a includes light source 110, condenser lens 120, reflector 130a and channel pipe 140.

As illustrated in FIGS. 6A to 6D, reflector 130a according to the modification includes reflection surface 131a, first recess 132 and second recess 133.

Reflection surface 131a according to the modification is formed larger than reflection surface 131 in embodiment 1. Specifically, reflection surface 131a according to the modification is about twice as large as reflection surface 131 of embodiment 1 in a direction along second central axis CA2 of reflector 130a. The shape of reflection surface 131a in a cross section including second central axis CA2 is linear.

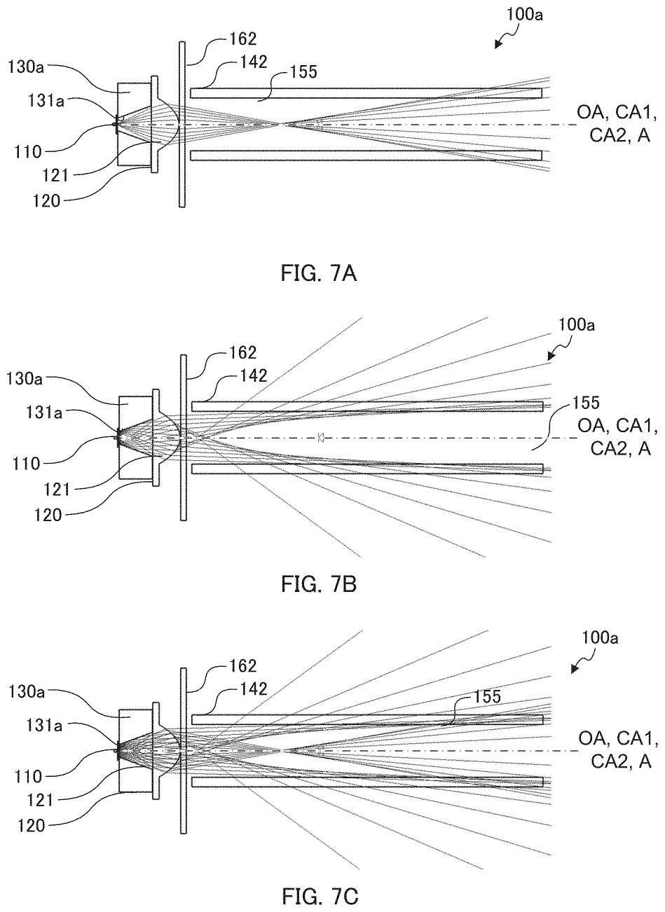

FIGS. 7A to 7C illustrate optical paths in ultraviolet sterilization apparatus 100a. FIG. 7A illustrates optical paths of light emitted from light source 110 and controlled by condenser lens 120 without controlled by reflector 130a, FIG. 7B illustrates optical paths of light emitted from light source 110 and controlled by reflector 130a, and FIG. 7C illustrates optical paths combining the optical paths of FIGS. 7A and 7B. FIGS. 7A to 7C illustrate only light source 110, condenser lens 120, reflector 130a and treatment channel 155 to show the optical paths.

As illustrated in FIGS. 7A and 7C, part (first part) of ultraviolet radiation emitted from light source 110, i.e., ultraviolet radiation having a small emission angle, directly reaches condenser lens 120 without controlled by reflector 130a. The ultraviolet radiation reaching condenser lens 120 enters condenser lens 120 from the flat surface of condenser lens 120 on the light source 110 side. The ultraviolet radiation entering condenser lens 120 is emitted to the outside from convex lens surface 121 on the channel pipe 140 side. During the procedure, the ultraviolet radiation is refracted by convex lens surface 121 so as to be condensed on optical axis OA of light source 110. The ultraviolet radiation emitted from convex lens surface 121 radiates through transparent plate 162 into treatment channel 155. Since treatment channel 155 is irradiated with the ultraviolet radiation condensed by condenser lens 120, a certain level of high illuminance can be obtained not only in the vicinity of light source 110 but also in the region far from light source 110.

As illustrated in FIGS. 7B and 7C, another part (second part) of ultraviolet radiation emitted from light source 110, i.e., ultraviolet radiation having a large emission angle, directly reaches reflection surface 131a of reflector 130a. The ultraviolet radiation having a large emission angle and reaching reflection surface 131a is reflected toward the incidence surface of condenser lens 210. The ultraviolet radiation reaching the incidence surface enters the condenser lens from the incidence surface and is emitted from convex lens surface 121. The ultraviolet radiation emitted from convex lens surface 121 radiates through transparent plate 162 into treatment channel 155. Ultraviolet sterilization apparatus 100a according to the modification has long optical paths from light source 110 to condenser lens 120, so that ultraviolet radiation enters condensing lens 120 at a small angle with respect to first central axis CA1. This small angle allows the ultraviolet radiation to reach the further back of treatment channel 155, thereby enhancing the illuminance in treatment channel 155.

(Effects)

From the foregoing, ultraviolet sterilization apparatus 100a according to the modification of embodiment 1 has the same effect as that of ultraviolet sterilization apparatus 100 according to embodiment 1.

### Embodiment 2

Ultraviolet sterilization apparatus 200 according to embodiment 2 differs from ultraviolet sterilization apparatus 100 according to embodiment 1 in the configuration around condenser lens 220. Therefore, the same components as those of ultraviolet sterilization apparatus 100 according to embodiment 1 are designated by the same reference numerals and the description thereof will be omitted.

(Configuration of Ultraviolet Sterilization Apparatus)

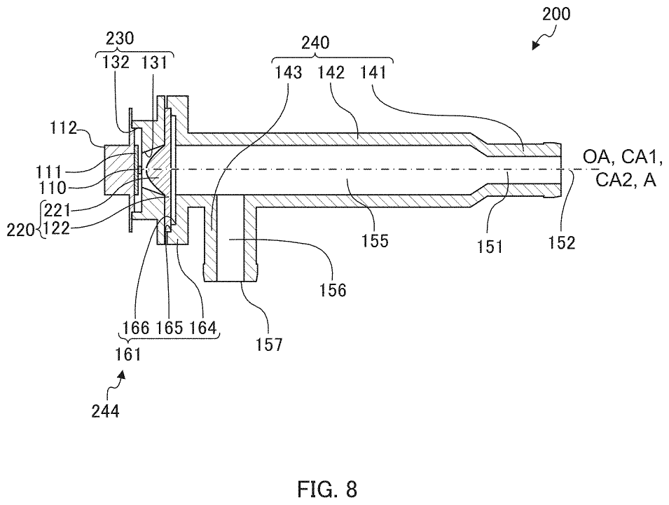

FIG. 8 is a cross-sectional view of ultraviolet sterilization apparatus 200 according to embodiment 2. As illustrated in FIG. 8, ultraviolet sterilization apparatus 200 includes light source 110, condenser lens 220, reflector 230 and channel pipe 240.

(Configuration of Condenser Lens)

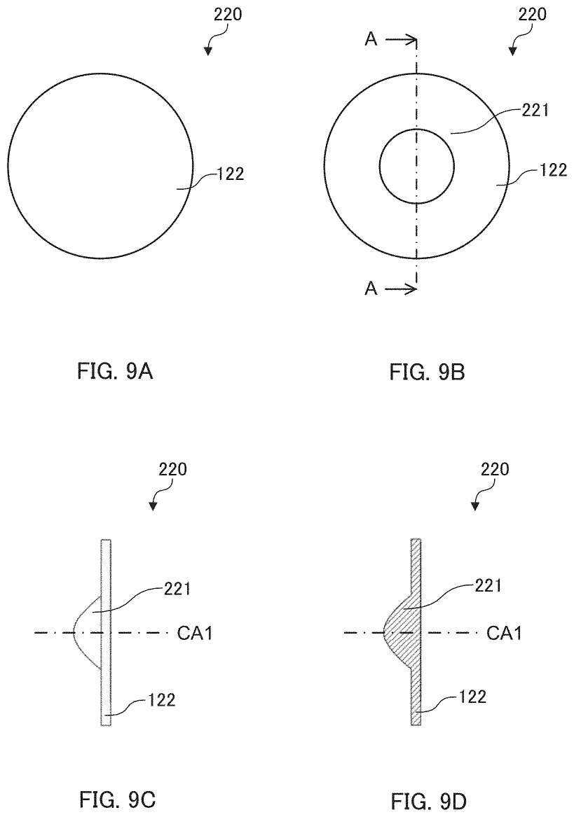

FIGS. 9A to 9D illustrate a configuration of condenser lens 220. FIG. 9A is a plan view of condenser lens 220, FIG. 9B is a bottom view thereof, FIG. 9C is a side view thereof and FIG. 9D is a cross-sectional view taken along line A-A shown in FIG. 9B.

As illustrated in FIGS. 9A to 9D, condenser lens 220 includes convex lens surface 221 and flange 122, and is circularly symmetrical with first central axis CA1 as a rotation axis. In a cross section including first central axis CA1, convex lens surface 221 is formed so that the length thereof in the direction perpendicular to first central axis CA1 decreases from the channel pipe 240 side to the light source 110 side. In the present embodiment, convex lens surface 221 is disposed on the light source 110 side, so that convex lens surface 221 functions as an incidence surface and the flat surface on the channel pipe 240 side functions as an emission surface.

Condenser lens 220 of the present embodiment functions also as transparent plate 162 in embodiment 1. Condenser lens 120 is disposed at reflector 130 in embodiment 1, but condenser lens 220 is disposed between reflector 230 and third recess 165 in the present embodiment.

(Configuration of Reflector)

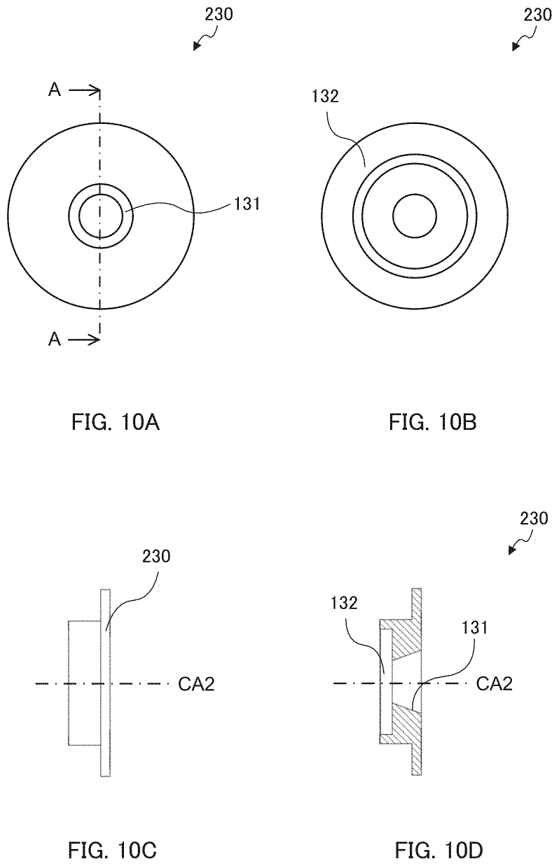

FIGS. 10A to 10D illustrate a configuration of reflector 230. FIG. 10A is a plan view of reflector 230, FIG. 10B is a bottom view thereof, FIG. 10C is a side view thereof and FIG. 10D is a cross-sectional view taken along line A-A shown in FIG. 10A.

Reflector 230 includes reflection surface 131 and first recess 132. In the present embodiment, reflector 230 functions also as fixing lid 163 in embodiment 1. The space surrounded by reflection surface 131 functions also as ultraviolet radiation transmitting hole 167 in embodiment 1. Fixing reflector 230 from the outside while condenser lens 220 is disposed at transparent plate holding portion 161 fixes condenser lens 220 between outer wall portion 164 and reflector 230. Convex lens surface 221 of condenser lens 220 is housed in the space surrounded by reflection surface 131.

(Configuration of Channel Pipe)

Channel pipe 240 includes inlet pipe 141, channel pipe body 142, outlet pipe 143 and incidence window 244. Incidence window 244 includes transparent plate holding portion 161. As described above, condenser lens 220 functions also as transparent plate 162, and reflector 230 functions also as fixing lid 163 in the present embodiment. Condenser lens 220 is disposed at third recess 165 of transparent plate holding portion 161. Reflector 230 is fixed to outer wall portion 164 of transparent plate holding portion 161.

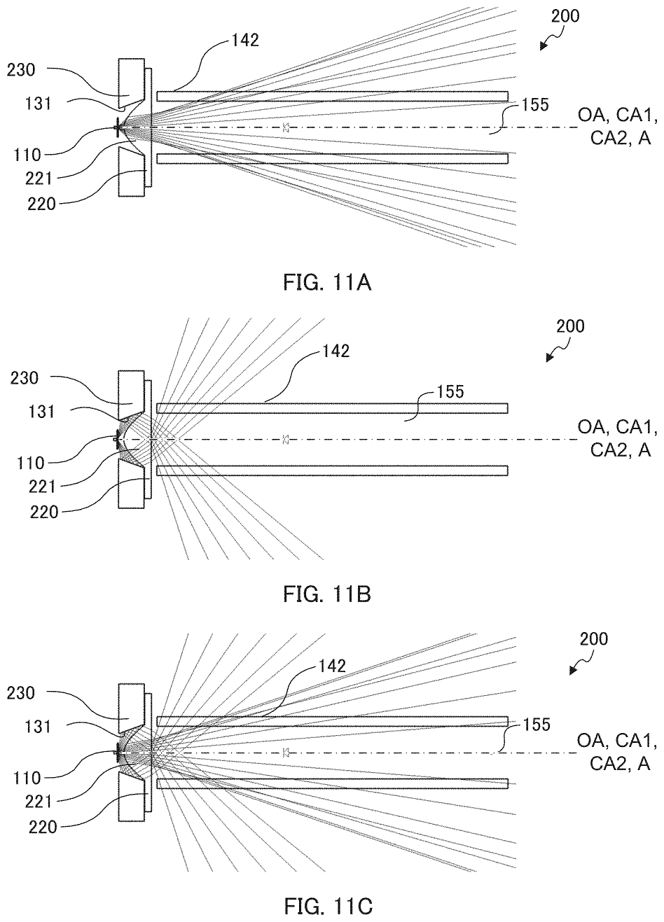

FIGS. 11A to 11C illustrate optical paths in ultraviolet sterilization apparatus 200. FIG. 11A illustrates optical paths of light emitted from light source 110 and controlled by condenser lens 220 without controlled by reflector 230, FIG. 11B illustrates optical paths of light emitted from light source 110 and controlled by reflector 230, and FIG. 11C illustrates optical paths combining the optical paths of FIGS. 11A and 11B. FIGS. 11A to 11C illustrate only light source 110, condenser lens 220, reflector 230 and treatment channel 155 to show the optical paths.

As illustrated in FIGS. 11A and 11C, part (first part) of ultraviolet radiation emitted from light source 110, i.e., ultraviolet radiation having a small emission angle, directly reaches condenser lens 220. The ultraviolet radiation reaching condenser lens 220 enters condenser lens 220 from convex lens surface 221 on the light source 110 side. During the procedure, the ultraviolet radiation is refracted by convex lens surface 221 toward optical axis OA of light source 110. The ultraviolet radiation thus emitted from light source 110 at a small emission angle is refracted by condenser lens 220 so that the angle of the ultraviolet radiation with respect to optical axis OA becomes smaller. The ultraviolet radiation entering condenser lens 220 is emitted to the outside from the flat surface on the treatment channel 155 side. The ultraviolet radiation emitted through convex lens surface 221 radiates into treatment channel 155. Since treatment channel 155 is irradiated with the ultraviolet radiation condensed by condenser lens 220, a certain level of high illuminance can be obtained not only in the vicinity of light source 110 but also in the region far from light source 110.

As illustrated in FIGS. 11B and 11C, another part (second part) of ultraviolet radiation emitted from light source 110, i.e., ultraviolet radiation having a large emission angle, directly reaches reflection surface 131 of reflector 230. The ultraviolet radiation reaching reflection surface 131 is reflected toward the incidence surface of condenser lens 220. The ultraviolet radiation reaching the incidence surface enters the condenser lens from convex lens surface 221 and is emitted from the flat surface. The ultraviolet radiation emitted from the flat surface radiates into treatment channel 155.

[Modification]

Ultraviolet sterilization apparatus 200a of a modification of embodiment 2 will be described. Ultraviolet sterilization apparatus 200a of the modification differs from ultraviolet sterilization apparatus 200 according to embodiment 2 only in the configuration of reflector 230a. Therefore, the same components as those of ultraviolet sterilization apparatus 200 according to embodiment 2 are designated by the same reference numerals and the description thereof will be omitted.

(Configuration of Ultraviolet Sterilization Apparatus)

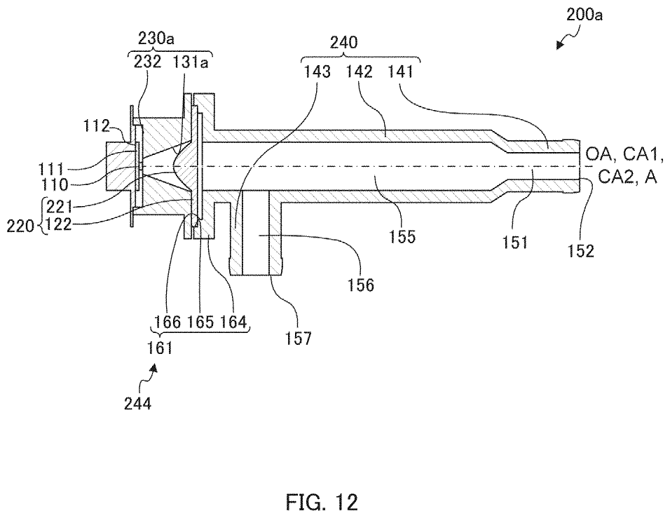

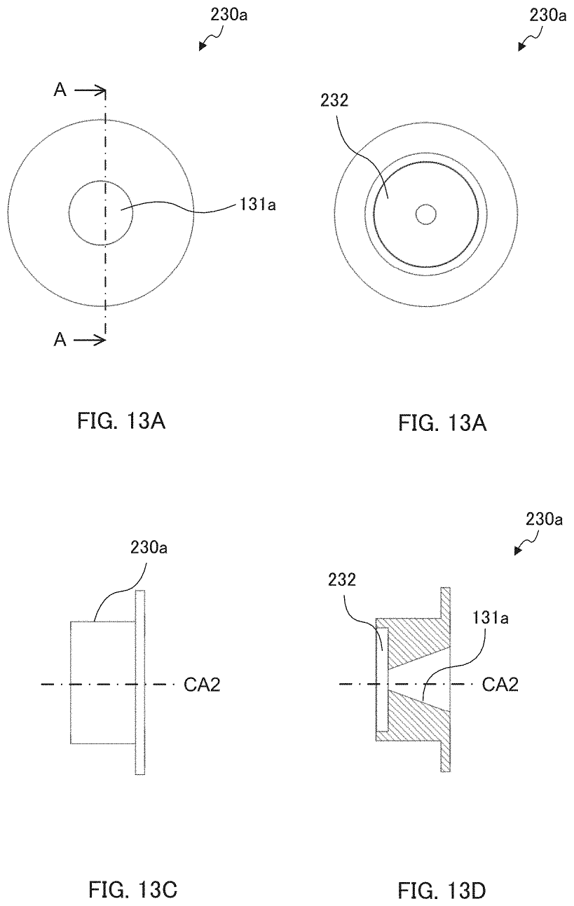

FIG. 12 is a cross-sectional view of ultraviolet sterilization apparatus 200a according to the modification. FIGS. 13A to 13D illustrate a configuration of reflector 230a. FIG. 13A is a plan view of reflector 230a, FIG. 13B is a bottom view thereof, FIG. 13C is a side view thereof and FIG. 13D is a cross-sectional view taken along line A-A shown in FIG. 13A.

As illustrated in FIG. 12, ultraviolet sterilization apparatus 200a includes light source 110, condenser lens 220, reflector 230a and channel pipe 240.

As illustrated in FIGS. 13A to 13D, reflector 230a of the modification includes reflection surface 131a and first recess 232.

Reflection surface 131a in the modification is formed larger than reflection surface 131 in embodiment 1. Specifically, reflection surface 131a according to the modification is about twice as large as reflection surface 131 of embodiment 1 in a direction along second central axis CA2 of reflector 230a. The shape of reflection surface 131a in a cross section including second central axis CA2 is linear.

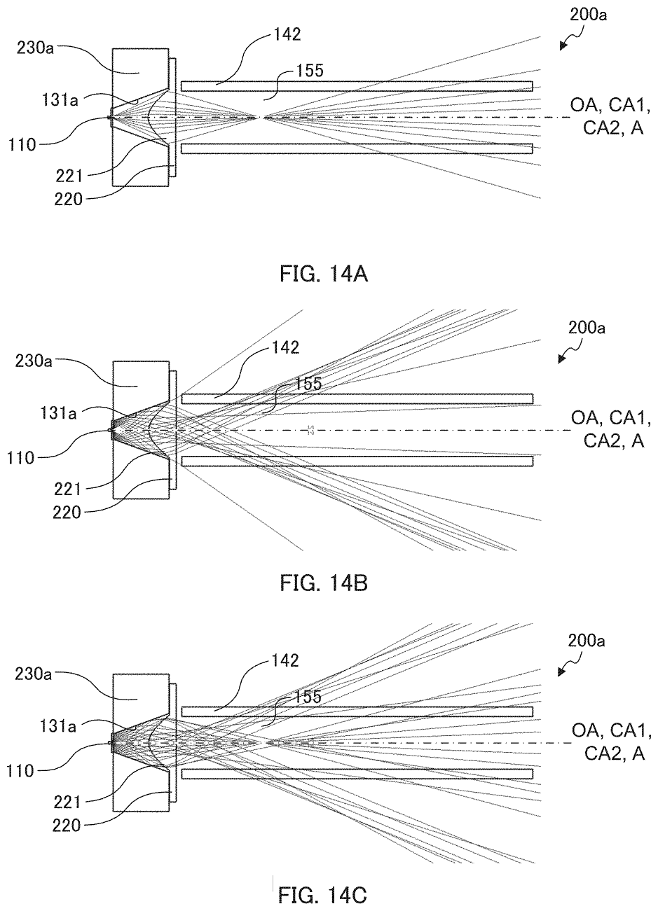

FIGS. 14A to 14C illustrate optical paths in ultraviolet sterilization apparatus 200a. FIG. 14A illustrates optical paths of light emitted from light source 110 and controlled by condenser lens 220 without controlled by reflector 230, FIG. 14B illustrates optical paths of light emitted from light source 110 and controlled by reflector 230a, and FIG. 14C illustrates optical paths combining the optical paths of FIGS. 14A and 14B. FIGS. 14A to 14C illustrate only light source 110, condenser lens 220, reflector 230a and treatment channel 155 to show the optical paths.

As illustrated in FIGS. 14A and 14C, part (first part) of ultraviolet radiation emitted from light source 110, i.e., ultraviolet radiation having a small emission angle, directly reaches condenser lens 220. The ultraviolet radiation reaching condenser lens 220 enters condenser lens 220 from convex lens surface 221 on the light source 110 side. During the procedure, the ultraviolet radiation is refracted by convex lens surface 221 so as to be condensed on optical axis OA of light source 110. The ultraviolet radiation entering condenser lens 220 is emitted from the flat surface to the outside. The ultraviolet radiation emitted from the flat surface radiates into treatment channel 155. Since treatment channel 155 is irradiated with the ultraviolet radiation condensed by condenser lens 220, a certain level of high illuminance can be obtained not only in the vicinity of light source 110 but also in the region far from light source 110.

As illustrated in FIGS. 14B and 14C, another part (second part) of ultraviolet radiation emitted from light source 110, i.e., ultraviolet radiation having a large emission angle, directly reaches reflection surface 131a of reflector 230. The ultraviolet radiation reaching reflection surface 131a is reflected toward the incidence surface of condenser lens 220. The ultraviolet radiation reaching the incidence surface enters the condenser lens from convex lens surface 221 and is emitted from the flat surface. The ultraviolet radiation emitted from the flat surface radiates into treatment channel 155.

(Effects)

From the foregoing, the present invention is capable of reducing the number of parts constituting ultraviolet sterilization apparatuses 200 and 200a in addition to having the effect of embodiment 1. The manufacturing cost thus can be reduced.

In addition, treatment channel 155 may be irradiated with ultraviolet radiation while light source 110 is rotated to increase uniformity of illuminance distribution of the ultraviolet radiation emitted from light source 110, thereby more uniformly sterilizing a fluid in treatment channel 155.

This application claims priority based on Japanese Patent Application No. 2018-023989 filed on Feb. 14, 2018, the entire contents of which including the specification and the drawings are incorporated herein by reference.

### INDUSTRIAL APPLICABILITY

The present invention is advantageous for an ultraviolet sterilization apparatus to sterilize, for example, clean water or an agricultural fluid.

### REFERENCE SIGNS LIST

100, 100a, 200, 200a Ultraviolet sterilization apparatus
110 Light source
120, 220 Condenser lens
130, 130a, 230, 230a Reflector
140, 240 Channel pipe
111 Substrate
112 Base
121, 221 Convex lens surface
122 Flange
131, 131a Reflection surface
132, 232 First recess
133 Second recess
141 Inlet pipe
142 Channel pipe body
143 Outlet pipe
144, 244 Incidence window
151 Inlet channel
152 Inlet port
155 Treatment channel
156 Outlet channel
157 Outlet port
161 Transparent plate holding portion
162 Transparent plate
163 Fixing lid
164 Outer wall portion
165 Third recess
166 Fourth recess
167 Ultraviolet radiation transmitting hole
300 Ultraviolet irradiation apparatus
A Axis
CA1 First central axis
CA2 Second central axis
OA Optical axis

1. An ultraviolet sterilization apparatus for sterilizing a fluid flowing through a treatment channel by irradiating the fluid with ultraviolet radiation, the ultraviolet sterilization apparatus comprising:
a channel pipe including therein the treatment channel;
a light source configured to emit the ultraviolet radiation;
a condenser lens configured to condense, toward the treatment channel, a part of the ultraviolet radiation emitted from the light source; and
a reflector configured to reflect, toward the treatment channel, another part of the ultraviolet radiation emitted from the light source.

2. The ultraviolet sterilization apparatus according to claim 1, wherein:
the condenser lens is disposed between the channel pipe and the light source; and
the condenser lens includes a convex lens surface on a side of the channel pipe or the light source.

3. The ultraviolet sterilization apparatus according to claim 1, wherein:
the treatment channel is formed to be linear; and
an optical axis of the light source, a first central axis of the condenser lens, a second central axis of the reflector, and an axis of the treatment channel are coincident with each other.

4. An ultraviolet irradiation apparatus for sterilizing a fluid flowing through a treatment channel by irradiating the fluid with ultraviolet radiation, the ultraviolet irradiation apparatus comprising:
a light source configured to emit the ultraviolet radiation;
a condenser lens configured to condense, toward the treatment channel, a part of the ultraviolet radiation emitted from the light source; and
a reflector configured to reflect, toward the treatment channel, another part of the ultraviolet radiation emitted from the light source.

5. The ultraviolet sterilization apparatus according to claim 2, wherein:
the treatment channel is formed to be linear; and
an optical axis of the light source, a first central axis of the condenser lens, a second central axis of the reflector, and an axis of the treatment channel are coincident with each other.
